(19) United States
(12) Patent Application Publication
(10) Pub. No.: US 2005/0169847 A1
(43) Pub. Date: Aug. 4, 2005

(54) Process for the treatment of particles for use in pharmaceutical formulations

(76) Inventor: Robert Price, Chepstow (GB)

Correspondence Address: BELL & ASSOCIATES, 416 FUNSTON ST., SUITE 100, SAN FRANCISCO, CA, 94118, US

(21) Appl. No.: 10/513,772

(22) PCT Filed: May 6, 2003
(86) PCT No.: PCT/GB03/01894

(30) Foreign Application Priority Data
May 8, 2002 (GB) 02 10527.8

(51) Int. Cl. A61L 9/04; A61K 9/14

(52) U.S. Cl. 424/46

(57) ABSTRACT

A process for the surface treatment of particles suitable for use in a pharmaceutical formulation, which comprises preparing in a temperature controlled environment a saturated solution of the substance of which the particles to be treated are composed; suspending the particles to be treated in said saturated solution; adjusting the temperature to a predetermined level in the direction of increased solubility of said substance; and harvesting the treated particles.

This invention relates to an improved process for the treatment of particles for use in pharmaceutical formulations, especially for inhalation formulations.

A number of drugs are available to treat patients with asthma or other respiratory disorders. One preferred way of administering such drugs is by inhalation, and a number of different types of inhalers, specifically metered dose inhalers, nebulisers, and dry powder inhalers, are available.

The efficacy of administering drugs by inhalation depends critically upon the physical properties of the particles being inhaled. In general, drug particles of relatively small particle size are carried by carrier particles of a much larger particle size. On inhalation, the smaller drug particles are stripped off from the carrier particles and carried into the lungs. The carrier particles remain in the back of the throat. This mechanism tends to be rather inefficient, with only a relatively small proportion of the drug particles reaching the lungs.

The surface texture of the particles used in an inhalation formulation plays a crucial role in the behaviour of the particles and hence upon the clinical effectiveness of the formulation. The shape and texture of the carrier particle should be such as to give sufficient adhesion force to hold the drug particles to the surface of the carrier particles during manufacture of the formulation and storage in the inhaler, but such as to allow the dispersion of the drug particles into the lungs on inhalation.

In the case of known carriers, the surface rugosity of the dominant crystalline face of a commercial grade carrier particle is usually rough on a micrometer scale, with an array of asperities and clefts in its surface. The site of an asperity or a cleft is believed to be an area of high surface free energy. The drug particles are preferentially attracted to and adhere more strongly to high energy sites causing large variability and potentially reduced dispersibility of the drug particles from the carrier surface.

Methods are known for producing perfectly smooth particles. Colombo et al, Respiratory Drug Delivery VII, 2000, 629-631, and WO 01/05429, describe a technique for smoothing the surface of lactose particles for use in inhalation mixtures. In this method, lactose particles are physically modified in the bowl of a high-speed mixer, with successive steps of wetting with a solvent and drying under vacuum. Such a physical smoothing method is claimed to be capable of producing perfectly smooth particles. It is not, however, susceptible to precise control and hence cannot give particles with a carefully defined surface rugosity, and would be very time-consuming to carry out on an industrial scale.

Further, carrier particles with completely smooth surfaces present their own difficulties. Drug particles bind too strongly to perfectly smooth surfaces and hence they are difficult to dislodge from the carrier particles when the formulation is inhaled.

It would therefore be highly advantageous to dramatically decrease the degree of high surface free energy sites on conventional carrier particles without producing a perfectly smooth particle, and to produce particles with well-defined surface roughness on a nanometer scale.

A number of techniques are known for crystallising particles out of solution under conditions such that the crystals have particular surface properties. For example, WO 99/48475 describes a method of preparing crystals of particles suitable for use in inhalation which comprises crystallising particles from a system in which the viscosity of the medium is adjusted and controlled.

U.S. Pat. No. 5,376,386 describes and claims a particulate carrier suitable for use in inhalers having an average particle size of from 5 to 1000 microns and a rugosity of less than 1.75. Such particles are made by a process of crystallisation from a saturated aqueous solution by the addition of a water immiscible organic solvent and a solvent which is miscible with both water and the immiscible solvent and controlling the conditions under which crystallisation and crystal growth occurs.

Such crystallisation processes have however generally proved to be unsatisfactory, as crystallisation is inherently difficult to control. Because of this, alternative approaches to the problem of optimising inhalation formulations have been proposed. One such approach is described in U.S. Pat. No. 6,153,224. Here, additive particles are attached to the surfaces of the carrier particles to promote the release of the drug particles from the carrier particles. Such a process significantly increases the cost and complexity of the formulation process.

The present invention has as its objective the provision of a process for the treatment of particles suitable for use in pharmaceutical formulations which avoids various of the disadvantages of prior art processes.

Accordingly the present invention provides a process for the surface treatment of particles suitable for use in a pharmaceutical formulation, which comprises the steps of:
i) preparing in a temperature controlled environment a saturated solution of the substance of which the particles to be treated are composed;
ii) suspending the particles to be treated in said saturated solution;
iii) adjusting the temperature to a predetermined level in the direction of increased solubility of said substance; and
iv) harvesting the treated particles.

The process of the present invention may be applied to any soluble particles suitable for use in a pharmaceutical formulation, and is especially valuable when applied to carrier particles or to drug particles for use in inhalation formulations. Preferably the particles to be treated are formed primarily of crystalline material. Known techniques for the manufacture or treatment of crystalline particles such as high energy milling or micronisation induce varying degrees of disorder to the surfaces of the processed particles of crystalline material. Such processes generate amorphous disorder in the nominally crystalline material, such amorphous regions being mainly concentrated at the surface. The process of the invention will preferentially aid in the removal of the thermodynamically unstable amorphous regions, leading to an enhanced stability of the crystalline particles.

Carrier particles prepared by the process of the invention for use in inhaler formulations preferably have a median diameter in the range of from 50 to 250 microns, preferably 63 to 90 microns. Preferably at least 95% of the particles will be of a size which falls within these ranges.

With proper control of the process conditions, the process of the invention may if desired be carried out such that the overall size of the particles used as starting material in the process of the invention will not differ substantially from that of the harvested particles. Therefore in a preferred embodiment of the invention the particles to be treated by the process of the invention have a mean diameter in the range of from 50 to 250 microns, preferably 63 to 90 microns.

The process of the invention may be applied to any soluble carrier useful in inhaler compositions, for example monosaccharides such as lactose, glucose, fructose, mannitol, arabinose, xylitol and dextrose and their monohydrates, disaccharides such as trehalose, maltose or sucrose, and polysaccharides such as dextrins or dextrans. Especially preferred carriers include glucose, fructose, mannitol, sucrose and lactose and their monohydrates. Lactose and its monohydrate are particularly preferred.

The process of the invention may also advantageously be applied to drug particles for use in inhalation formulations. Such drug particles which may be prepared by the process of the invention preferably have a median diameter in the range of from 1 to 10 microns. Typical drugs include salbutamol sulphate, terbutaline sulphate, salmeterol xinafoate, fenoterol and formeterol, and corticosteroids such as fluticasone propionate, budesonide and beclomethasone. Another area of application for the process of the invention include particles for use in solid based suspensions for oral administration or inhalation, as well as for any powdered drug material. The process of the invention is expected to aid the maintenance of stability of drug powders during storage.

Steps (i) and (ii) of the process of the invention may be carried out as discrete stages, or they may be integrated into a single process step by taking a quantity of particles in excess of that required to be harvested in step (iv) of the process and adding these particles to a suitable solvent in such an amount that a certain proportion thereof will dissolve to form the saturated solution and the remaining particles will remain to be treated by step (iii) of the process.

The solvent used in the process of the invention will of course be chosen depending upon the identity of the particles to be treated. Preferably the particles to be treated are composed of a water soluble material and the solvent used in the process of the invention is water, optionally together with a co-solvent. Other solvents or solvent mixtures may however be used, for example alcohols or water/alcohol mixtures or any other suitable organic solvent.

In most cases, solubility increases with temperature, and therefore step (iii) of the process of the invention will be carried out by raising the temperature. As the temperature increases, the solution becomes able to dissolve more of the solid. The process may be regarded as a surface etching process in which asperities on the surface of the articles to be treated are selectively etched away as the temperature increases. Etching tends to take place preferentially at those sites having high surface free energy. Excessive etching and hence significant erosion of mass of the particles to be treated is avoided by careful temperature control. In some cases, the solubility of the solid material will decrease with temperature, and in this case step (iii) of the process of the invention will be carried out by lowering the temperature. Depending upon the various process parameters adopted including the temperature difference required, the temperature may be adjusted in stepwise fashion or in a single step.

Careful control of the various process parameters enables particles with a precisely defined surface rugosity to be obtained. The time taken to carry out the process of the invention will depend upon the results desired and upon the detailed design of the apparatus used. In general, the longer the processing time during which the crystals to be treated are in contact with the treating solution, the lower will be the surface rugosity of the crystals harvested at the end of the process. Stirring is preferably continually applied to the solution containing the particles being treated throughout the treatment process to maintain a stable situation in which the solution does not become supersaturated.

Preferably, the particles obtained by the process of the resent invention have a surface rugosity of between 1.05 and 1.80.

The process according to the invention may be carried out as a batch process or as a semi-continuous process. The equipment needed is simple and cheap and can readily be constructed as an add-on to existing equipment for the preparation of particles for use in pharmaceutical formulations, or for the preparation of pharmaceutical formulations.

With a knowledge of the temperature dependency of the solubility of the substance to be treated, it is possible by careful control of temperature in the process according to the invention to achieve the precise degree of etching of the particles required. The following expression (%σ) gives the percentage difference between the solute concentration at temperature T and the solute concentration at the saturation temperature:
−%σ=100×(C_{T}−C_{S})/C_{S}
where C_{T} is solubility at the etching temperature and C_{S} is the solubility at the saturation temperature. It is also possible to define an equation which describes the solubility versus temperature profile; for α-lactose monohydrate the equation is:
S_{T}=19.892−0.2937T+0.0138T²+0.00003T³
where S_{T} is the solubility of lactose in mg/100 ml water and T is the temperature of the solution in ° C.

Thus, if the saturation temperature, etching temperature, volume of the liquid phase and the amount of material added to the system is known the whole etching process of any particle surface can be accurately determined and controlled. The process of the present invention avoids the difficulties inherent in crystallisation techniques. Such techniques are inherently difficult to control and their results are difficult to predict, as every student who has tried to induce crystallisation in a test-tube will testify. Unlike crystallisation, the kinetics of dissolution are rapid. Furthermore, once saturation conditions within the treatment solution are re-established following a temperature change, further dissolution and crystallisation are inhibited. This means that the surface etching process of the present invention is extremely easy to control and predictable and consistent results can readily be achieved between batches.

The particles prepared by the process of the invention may be harvested by any suitable method, typically filtration. They may then be washed with a non-solvent, sieved, and dried, ready for further processing if required to prepare a pharmaceutical formulation.

The present invention avoids the problems associated with the production of crystals by crystallisation from a solution and, because it may use as starting material any commercial-grade particles, for example commercial grade lactose, it is very economical in use. It may be readily applied on an industrial scale, and adds only a small number of additional steps to current industrial processes for the production of particles for pharmaceutical formulations, especially inhalation formulations. For these reasons it is more cost effective than other proposals to improve the preparation of such formulations.

The process of the invention is especially useful for the preparation of particles for use in inhaler formulations. Such particles may be carrier particles or drug particles of both. Thus in a further aspect, the present invention provides a process for the preparation of an inhaler formulation comprising drug particles and carrier particles, which comprises the steps of treating carrier particles by process steps i) to iv) as defined above; washing and drying the thus treated carrier particles; and blending said carrier particles with said drug particles.

In yet a further aspect, the present invention provides a process for the preparation of an inhaler formulation comprising drug particles and carrier particles, which comprises the steps of treating drug particles by process steps i) to iv) as defined above; washing and drying the thus treated drug particles; and blending said drug particles with said carrier particles.

In both these aspects, an especially preferred embodiment is when the carrier particles comprise lactose and the drug particles comprise salbutamol.

The following Examples illustrate the invention. In the Figures, the white bars indicate the scale, the length of the bar being equivalent to 10 μm.

### EXAMPLE 1

Inhalation grade α-lactose (Lactochem (Trade mark), available from Borculo Whey) was sieve fractioned in the particle size range 63-90 microns. A saturated solution of α-lactose monohydrate in water was prepared and continually stirred at a constant temperature of 25° C. Temperature within the vessel was controlled to within 0.1° C. via a refrigerated controlled water bath. 100 ml of the saturated solution was subsequently filtered into a dissolution vessel, which was maintained at the saturation temperature, and 50 g of sieved (63-90 microns) α-lactose monohydrate was added to the saturated solution in the dissolution vessel.

The temperature within the dissolution vessel was increased by 5° C. and the mixture was stirred for a period of 30 minutes. The mixture was then filtered, washed with cyclohexane, sieved to retrieve particles in the size range 63 to 90 microns, and dried.

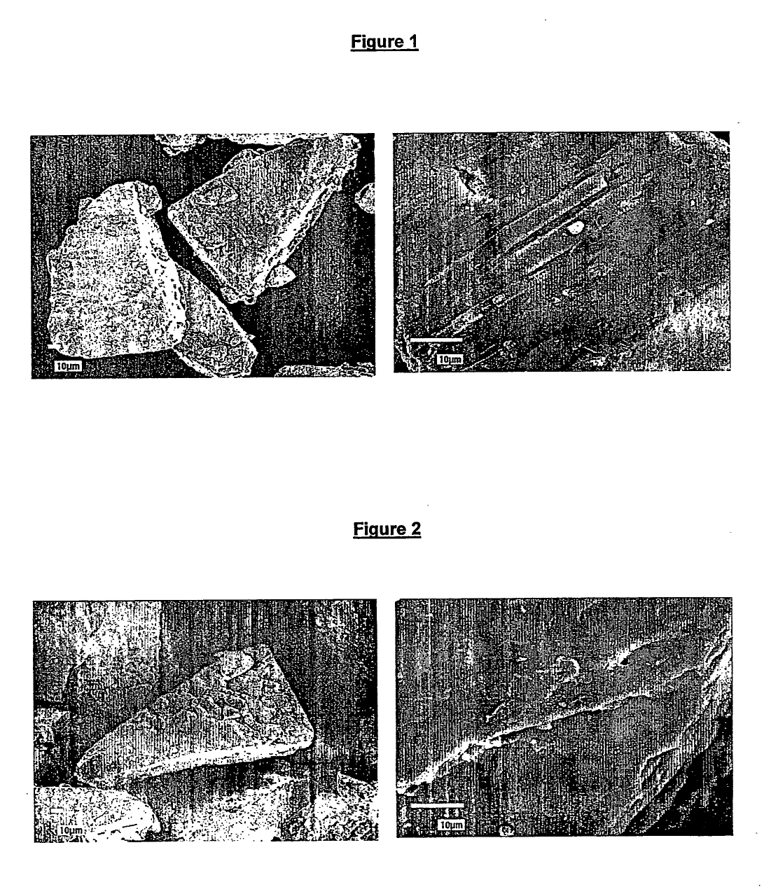

Representative scanning electron microscopy (SEM) topographical images of the resulting lactose particles are shown in FIG. 1.

### EXAMPLES 2 TO 5

The method of Example 1 was repeated except that the temperature within the dissolution vessel was raised by differing amounts. Representative SEM images of the resulting lactose particles are shown in FIGS. 2 to 5.

For comparison, representative SEM images of the untreated α-lactose monohydrate used as starting material in Examples 1 to 5 are shown in FIG. 6. A clear reduction in surface rugosity of the particles treated in accordance with Example 1 to 5 compared with the untreated particles can be seen.

### EXAMPLE 6

### In Vitro Testing of Salbutamol Availability

Physical blends of micronised salbutamol sulphate and particles of α-lactose monohydrate (untreated and treated in accordance with Examples 1 to 5) in a ratio of 1:67.5 w/w were prepared by mixing in a turbula mixer for 30 minutes. These mixtures were then loaded into gelatin capsules for a monohaler device for in vitro measurements. Each capsule contained a 400 microgram dose of salbutamol sulphate. In vitro aerosol deposition was performed using a twin stage impinger by the standard method described in European Pharmacopaeia 1997 at 60 L/min. This method models the aerosolisation behaviour of a dry powder inhaler formulation. The fine particle fraction is calculated as the percentage of the total amount of drug emitted from the device that reaches the lower stage (stage 2) of the twin stage impinger (the particle “cut-off” diameter for the device at 60 L/min is dae less than 6.4 micron—i.e. particles of greater than this diameter are not permitted to pass to the lower stage). The fine particle fraction provides an indication of the proportion of the drug particles which would reach the deep lung in a patient, and is hence a measure of the pharmaceutical efficacy of an inhalation formulation. Measurements were performed in replicates of three. Drug analysis was performed by fluorescence spectroscopy.

The results for the untreated and treated lactose surfaces are shown in the following Table. The data in the Table shows the average salbutamol sulphate content in micrograms recovered from each stage of the twin stage impinger, and the fine particle fraction calculated from this measurement.

The results show that use of lactose particles treated by the process of the present invention results in an increase in the deposition of salbutamol particles in the lower stage of the twin stage impinger, which indicates a significantly improved availability of the drug in vivo.

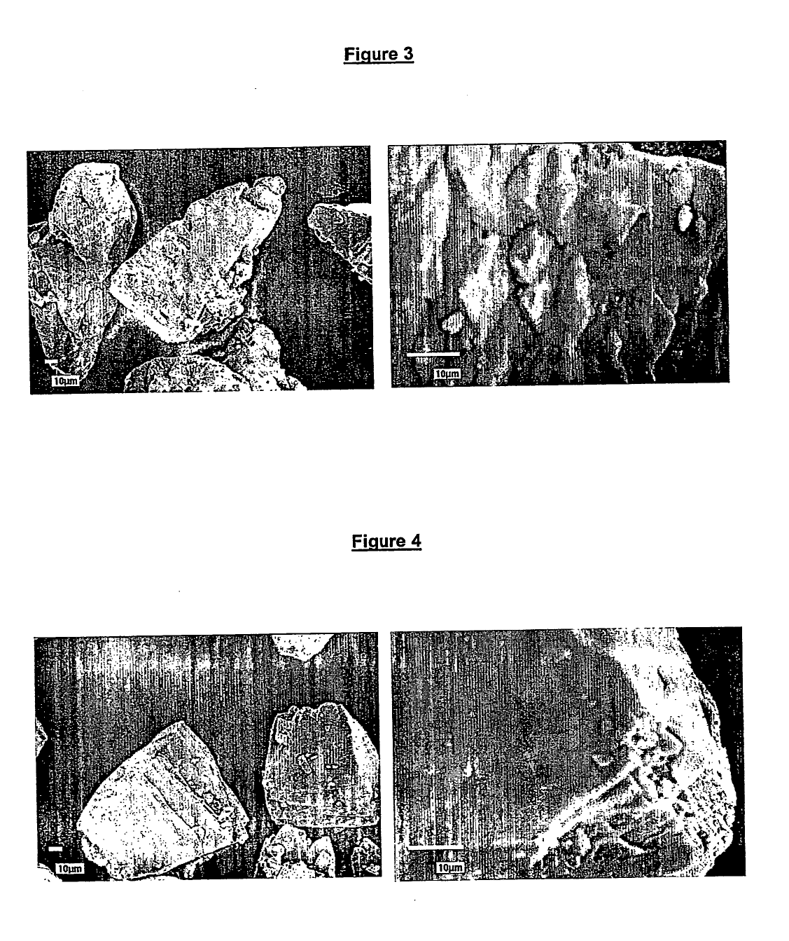

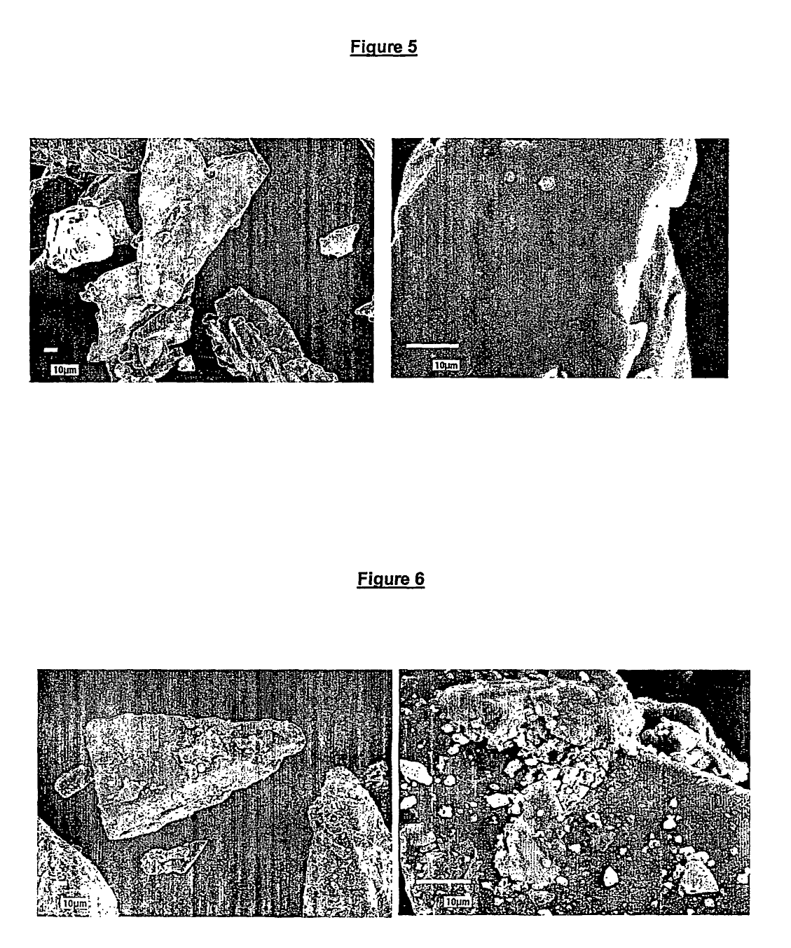

TABLE

| | | Weight of particles | | |
|---|---|---|---|---|
| | | (less than 6.4 μm) | | |
| | | reaching stages 1 and | | |
| | | 2 of the twin stage | | |
| Example | Etching | impinger | | Fine particle |
| No. | Conditions | Stage 1 | Stage 2 | fraction (%) |
| 1 | 5° C. etch | 137.7 | 79.35 | 25.3 |
| (FIG. 1) | | | | |
| 2 | 10° C. etch | 202.5 | 86.4 | 19.7 |
| (FIG. 2) | | | | |
| 3 | 15° C. etch | 159.4 | 123.6 | 33.3 |
| (FIG. 3) | | | | |
| 4 | 20° C. etch | 161.4 | 93.3 | 24.8 |
| (FIG. 4) | | | | |
| 5 | 25° C. etch | 201.0 | 100.0 | 23.5 |
| (FIG. 5) | | | | |
| Comparison | Untreated | 199.5 | 47.4 | 13.9 |
| (FIG. 6) | particles | | | |

1. A process for the surface treatment of particles suitable for use in a pharmaceutical formulation, comprising the steps of: i) preparing in a temperature controlled environment a saturated solution of the substance of which the particles to be treated are composed; ii) suspending the particles to be treated in said saturated solution; iii) adjusting the temperature to a predetermined level in the direction of increased solubility of said substance; and iv) harvesting the treated particles.

2. The process according to claim 1, wherein the particles to be treated are carrier particles or drug particles for use in inhalation formulations.

3. The process according to claim 1, in which the particles to be treated are carrier particles having a median diameter in the range of from 63 to 90 microns.

4. The process according to claim 1 in which the particles to be treated are lactose or its monohydrate.

5. The process according to claim 1, in which the particles to be treated are drug particles having a median diameter in the range of from 1 to 10 microns.

6. The process according to claim 1, in which the particles to be treated are composed of a water soluble material and the solvent used in the process of the invention comprises water.

7. The process according to claim 1, in which the particles produced by the process have a surface rugosity of between 1.05 and 1.80.

8. A process for the preparation of an inhaler formulation comprising drug particles and carrier particles, the process comprising the steps of: i) preparing in a temperature controlled environment a saturated solution of the substance of which the particles to be treated are composed; ii) suspending the particles to be treated in said saturated solution; iii) adjusting the temperature to a predetermined level in the direction of increased solubility of said substance; and iv) harvesting the treated particles; v) washing and drying the thus treated carrier particles; and vi) blending said carrier particles with drug particles.

9. A process for the preparation of an inhaler formulation comprising drug particles and carrier particles, the process comprising the steps of: i) preparing in a temperature controlled environment a saturated solution of the substance of which the particles to be treated are composed; ii) suspending the particles to be treated in said saturated solution; iii) adjusting the temperature to a predetermined level in the direction of increased solubility of said substance; and iv) harvesting the treated particles; v) washing and drying the thus treated drug particles; and vi) blending said drug particles with carrier particles.

10. The process as claimed in claim 8, in which the carrier particles comprise lactose or its monohydrate and the drug particles comprise salbutamol.

11. A process as claimed in claim 9, in which the carrier particles comprise lactose or its monohydrate and the drug particles comprise salbutamol.
